(19) United States
(12) Patent Application Publication
(10) Pub. No.: US 2023/0263618 A1
(43) Pub. Date: Aug. 24, 2023

(54) Covered Stent and Covered Stent System for Implantation at Vascular

(71) Applicant: Forever Medical (Suzhou) Co., Ltd., Suzhou City (CN)

(72) Inventor: Xiaoming Zhang, Suzhou City (CN)

(73) Assignee: Forever Medical (Suzhou) Co., Ltd., Suzhou City (CN)

(21) Appl. No.: 18/003,003

(22) PCT Filed: Jul. 5, 2021
(86) PCT No.: PCT/CN2021/104487

§ 371 Date: Dec. 22, 2022

(30) Foreign Application Priority Data
Jul. 3, 2020 (CN) 202010638641.5

(51) Int. Cl. A61F 2/07

(52) U.S. Cl. CPC A61F 2/07; A61F 2002/0081

(57) ABSTRACT

A covered stent for implantation at vascular branches includes a stent body and a membrane fixedly connected to the stent body, wherein micropore areas are provided on the membrane, and the micropore areas have a plurality of micropores which are arranged into an array with pore spacing ranging from 0.2 mm to 2.0 mm. The micropores are capable of allowing blood flow to pass through, and during implantation of the covered stent, the micropore areas are used to identify branch regions of blood vessels and provide an access path for implantation of branch stents, such that the blood flow of nearby branch vessels will not be blocked while treating aortic lesions.

### CROSS-REFERENCE TO RELATED APPLICATIONS

This application is a National Stage of International Application No. PCT/CN2021/104487, filed Jul. 5, 2022, which claims priority to Chinese Patent Application No. 202010638641.5, filed Jul. 3, 2021, both of which are hereby incorporated by reference in their entireties.

### TECHNICAL FIELD

The present application relates to the field of medical devices, and in particular, to a covered stent and a covered stent system for implantation at vascular branches.

### BACKGROUND OF THE INVENTION

High blood pressure, connective tissue disease, and chest trauma may all lead to aortic lesions. Endovascular repair has become a reliable option for treating various aortic lesions. As aortic diseases involving branch vessels (such as aneurysms, arterial dissection, arterial penetrating ulcers, etc.) become more and more serious, and the importance of the reconstruction of blood supply of branch vessels is gradually discovered, the problem of how to effectively treat aortic lesions without affecting the blood supply of the branch vessels near the lesion has become the focus of attention in the interventional treatment operations for aortic lesions.

Currently, the clinical interventional techniques for treating aortic branch lesions mainly include a “chimney” technique, a branch stent technique, and an in-situ fenestration technique and the like. The main defects of the above three treatment techniques are: 1. A chimney stent will affect the adherence of a covered stent to the aorta, which is easy to cause endoleak. In addition, the aortic covered stent will compress the chimney stent, which may easily cause the cross section of the chimney stent channel to become smaller or occluded, and the long-term patency rate is low. 2. The positioning of branch stents is difficult, and the operation is difficult to perform and is time consuming; the alignment of the branch stents is incorrect, and the long-term patency rate of the branch vessels is low. 3. The main defect of the in-situ fenestration technique is: before membrane rupture and fenestration are performed, the membrane will completely block blood supply of the branch vessels, which may cause serious intraoperative complications and even lead to the death of a patient. In this case, extracorporeal circulation needs to be established in advance before the operation can be performed, which increases the complexity and operation time of the operation. In addition, when a balloon is used to dilate the window, it is easy to cause the membrane to tear in the direction of the weaving pattern, resulting in endoleak.

Therefore, according to the shortcomings of the existing treatment technology, it is of great significance to design a covered stent capable of retaining the blood flow of the branch vessels on the basis of treating aortic lesions.

### SUMMARY OF THE INVENTION

The purpose of the present application is to provide a covered stent and a covered stent system for vascular branches, which will not block the blood flow of nearby branch vessels while treating aortic lesions.

An object of the present application is achieved through the following technical solutions:

A covered stent for implantation at vascular branches, comprises a stent body and a membrane fixedly connected to the stent body, micropore areas are provided on the membrane, and the micropore areas have a plurality of micropores which are arranged into an array with pore spacing ranging from 0.2 mm to 2.0 mm, and the micropores are capable of allowing blood flow to pass through, and during implantation of the covered stent, the micropore areas are used to identify branch regions of blood vessels and provide an access path for implantation of branch stents.

In one embodiment, diameters of the micropores are in the range of 0.1 mm to 0.6 mm.

In one embodiment, the membrane is provided with one or more micropore areas, and the micropore areas are arranged in a range of 15 degrees to 360 degrees along the circumferential direction of the covered stent.

In one embodiment, the lengths of the micropore areas are in the range of 3 mm to 100 mm.

In one embodiment, the stent body is formed by weaving or laser cutting.

In a preferred embodiment, the stent body has sufficient supporting force, and the shape of the stent body is a straight cylinder structure or a tapered structure.

In a preferred embodiment, the stent body is partially covered, and the stent at the proximal end portion of the stent body is uncovered.

In a preferred embodiment, the stent body is fully covered.

In a preferred embodiment, the material of the stent body includes, but is not limited to, nickel-titanium alloy, cobalt-chromium alloy, magnesium alloy, 316L stainless steel, metal tantalum, polymer materials, and the like.

In one embodiment, a plurality of development marks are provided on the edge of the micropore areas to indicate the position of the micropore areas.

In a preferred embodiment, the development marks are attached to the edge of the micropore area by precision sewing or riveting.

In a preferred embodiment, the shapes of the development marks include, but are not limited to, circles, ovals, “8” shapes, and the like.

In a preferred embodiment, the number of the development marks is 2-10.

In a preferred embodiment, the material of the development marks includes, but is not limited to, platinum, gold, tantalum metal, medical stainless steel, and the like.

In one embodiment, the membrane is biocompatible, and the material of the membrane includes, but is not limited to, polytetrafluoroethylene, polyester, polyethylene terephthalate, polyurethane esters, and the like.

Another object of the present application is achieved through the following technical solutions:

A covered stent system for implantation at vascular branches, comprises a delivery system and a covered stent provided in the delivery system, a membrane breaker, a dilatation balloon and branch stents, and the covered stent comprises a stent body and a membrane fixedly connected to the stent body, micropore areas are provided on the membrane, and the micropore areas have a plurality of micropores which are arranged into an array with pore spacing ranging from 0.2 mm to 2.0 mm, and the micropores are capable of allowing blood flow to pass through, and when the covered stent is delivered to the target area by the delivery system and released, the blood flow through the micropores directs the micropore areas to be placed at the opening of the vascular branch, so the micropore areas can temporarily supply blood to the vascular branch, and the membrane breaker is used to penetrate the micropore areas to provide an opening for the dilation balloon to pass through, and the dilation balloon is used to further increase the size of the opening to facilitate the support of the branch stents through the micropore areas at the vascular branches to establish the blood supply channels of the vascular branches.

In one embodiment, the diameters of the micropores are in the range of 0.1 mm to 0.6 mm.

In a preferred embodiment, the membrane is provided with one or more micropore areas, and the micropores areas are arranged in a range of 15 degrees to 360 degrees along the circumferential direction of the covered stent.

In a preferred embodiment, the lengths of the micropore areas are in the range of 3 mm to 100 mm.

In one embodiment, a plurality of development marks are provided on the edge of the micropore areas to indicate the position of the micropore areas.

In a preferred embodiment, the development marks are attached to the edge of the micropore areas by precision sewing or riveting.

In a preferred embodiment, the shapes of the development marks include, but are not limited to, circles, ovals, “8” shapes, and the like.

In a preferred embodiment, the number of the development marks is 2-10.

In a preferred embodiment, the material of the development marks includes, but is not limited to, platinum, gold, tantalum metal, medical stainless steel, and the like.

In one embodiment, the stent body is formed by weaving or laser cutting.

In a preferred embodiment, the stent body has sufficient supporting force, and the shape of the bracket body is a straight cylinder structure or a tapered structure.

In a preferred embodiment, the material of the stent body includes, but is not limited to, nickel-titanium alloy, cobalt-chromium alloy, magnesium alloy, 316L stainless steel, metal tantalum, polymer materials, and the like.

In one embodiment, the membrane is biocompatible, and the material of the membrane includes, but is not limited to, polytetrafluoroethylene, polyester, polyethylene terephthalate, polyurethane esters, and the like.

Compared with the prior art, the present application has the following advantages:

1. The covered stent and covered stent system provided by the present application are provided with micropore areas on the membrane, and the micropore area have a plurality of micropores, which are capable of allowing blood flow to pass through; during the implantation of the covered stent, the micropore areas can identify the vascular branches area and temporarily supply blood to the vascular branches; moreover, a plurality of micropores arranged in the micropore areas are arranged into an array with pore spacing in the range of 0.2 mm to 2.0 mm, and the diameters of the micropores in the range of 0.1 mm to 0.6 mm; the setting of the micropore diameters and pore spacing can not only ensure the blood supply of the branch vessels, but also prevent the membrane from tearing during the puncture of the membrane breaker; even if the dilation balloon is further dilated, it only changes the size of the adjacent micropores in the micropore areas, but will not tear the membrane; therefore, the micropore areas can provide a good access indication and access window for the implantation of branch stents.

2. In the present application, a plurality of development marks are arranged on the edge of the micropore areas to indicate the position of the micropore areas. The micropore area section of the covered stent can be placed at the openings of the branch vessels through the development marks under the monitoring of Digital Subtraction Angiography (DSA), so that the covered stent will not block the blood flow of the branch vessels while treating aortic lesions; moreover, the development marks are also beneficial to the positioning of the branch stents, which can be released at the target position after passing through the micropore areas to establish a blood supply channel of the vascular branches.

3. The diameters of the micropores in the present application are in the range of 0.1 mm to 0.6 mm, and micropores that are not located at the opening of the vascular branches are attached to the blood vessel walls; due to the limitation of the micropore diameter and the characteristics of high blood flow rate and high pressure in the aorta, after a period of time, blood accumulation will appear in the micropores, causing the micropores to be permanently closed, so there is no need to worry about endoleak.

### BRIEF DESCRIPTION OF DRAWINGS

FIG. 1 is a schematic diagram of an embodiment of treating aortic lesions using the “chimney” technique in the prior art.

FIG. 2 is a schematic diagram of the structure of a covered stent for treating aortic lesions using the branch stent technique in the prior art.

FIG. 3 is a schematic diagram of the structure of a covered stent for treating aortic lesions using the in-situ fenestration technique in the prior art.

FIG. 4A is a schematic diagram of the structure of a membrane breaker in the present application.

FIG. 4B is a schematic diagram of the structure of a dilation balloon in the present application.

FIG. 4C is a schematic diagram of the structure of a branch stent in the present application.

FIG. 4D is a schematic diagram of the structure of a guide wire in the present application.

FIG. 4E is a schematic diagram of the structure of a delivery system in the prior art.

FIGS. 5A-5D are schematic diagrams illustrating one embodiment of the structure of a covered stent applicable to a vascular branch according to Embodiment 1 of the present application.

FIGS. 6A-6C are schematic diagrams illustrating another embodiment of the structure of a covered stent applicable to a vascular branch according to Embodiment 1 of the present application.

FIGS. 7A-7D are schematic diagrams illustrating the operation process of a covered stent system applicable to a vascular branch according to Embodiment 1 of the present application.

FIGS. 8A-8D are schematic diagrams illustrating one embodiment of the structure of a covered stent applicable to vascular branches according to Embodiment 2 of the present application.

FIGS. 9A-9D are schematic diagrams illustrating the operation process of a covered stent system applicable to vascular branches according to Embodiment 2 of the present application.

FIGS. 10A-10D are schematic diagrams illustrating one embodiment of the structure of a covered stent applicable to vascular branches according to Embodiment 3 of the present application.

FIGS. 11A-11D are schematic diagrams illustrating the operation process of a covered stent system applicable to vascular branches according to Embodiment 3 of the present application.

FIGS. 12A and 12B are schematic diagrams illustrating another embodiment of the structure of a covered stent applicable to vascular branches according to Embodiment 3 of the present application.

### DETAILED DESCRIPTION OF THE INVENTION

At The technical solutions brought by the technical solutions of the present application are further described in detail below with reference to the accompanying drawings and specific embodiments. The components in the drawings are not necessarily drawn to scale, emphasis instead being placed upon illustrating the concepts of the present application.

In the various embodiments of the present application, well-known structures or materials involved in are not described in detail. Furthermore, those skilled in the art should understand that the following various embodiments are only used for illustration, but not for limiting the protection scope of the present application.

Before describing the specific embodiments of the present application in detail, the terms involved in the present application are explained as follows:

“Micropore area” refers to an area defined by a plurality of micropores provided on a membrane, the plurality of micropores form an array according to different rules, and one array is one micropore area. The arrangement rules of each array can be the same or different. Pore sizes and/or pore spacing of the micropores in each array can be the same or different.

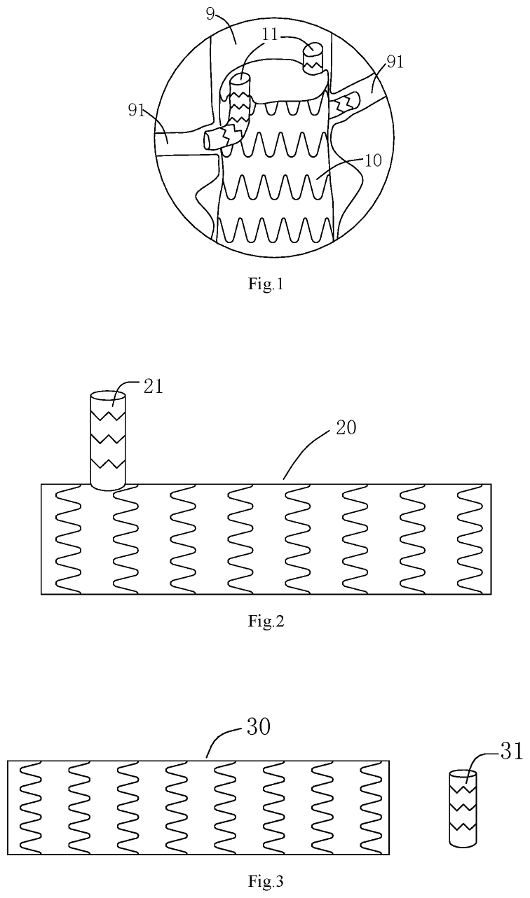

The “chimney” technique is a commonly used surgical technique for treating aortic lesions in the prior art. As shown in FIG. 1, the specific operation steps are as follows: firstly, under the monitoring of Digital Subtraction Angiography (DSA), a main stent 10 and chimney stents 11 are respectively delivered into an aorta 9 and corresponding vascular branches 91 without release; then, after the positioning of each stent is determined by digital subtraction angiography, the main stent 10 in the aorta cavity is first released, and then the chimney stents 11 are released between the main stent 10 and the aorta wall. Blood supply channels flowing to the branches are extruded out by the supporting force of the chimney stents 11, and in order to facilitate blood flow into the chimney stents 11, when releasing, the chimney stents 11 need to exceed the front edge of the main stent 10 by at least 5 mm.

The “branch stent” technique is a commonly used surgical technique for treating aortic lesions in the prior art. The structure of the branch covered stent is shown in FIG. 2. The main stent 20 and the branch stent 21 in the branch covered stent are generally of an integral structure. The specific operation steps of the “branch stent” technique are as follows: firstly, under the monitoring of Digital Subtraction Angiography (DSA), a guide wire passing through the branch stent 21 is pushed into a corresponding branch vessel, and a guide wire passing through the main stent 20 is pushed to the aortic lesions, then the main stent 20 is conveyed to the aortic lesions along its inner guide wire and the branch stent is ensured to be pre-placed at the opening of the branch vessel along its inner guide wire. After the positions of the stents are adjusted and confirmed by radiography, the main stent 20 and the branch stent 21 are released to isolate the aortic lesions and maintain the blood flow of the branch vessel.

The “fenestration” technique is a commonly used surgical technique for treating aortic lesions in the prior art. FIG. 3 illustrates the structure of a covered stent for using in the in situ fenestration technique. In this structure, a main stent 30 and a branch stent 31 of a covered stent are of a separate structure. The specific operation steps of the covered stent are as follows: firstly, under the monitoring of Digital Subtraction Angiography (DSA), the main stent 30 of the covered stent is placed in the aorta cavity of the diseased aorta, and at this time, a membrane at the opening of a branch vessel can completely block the blood flow of the branch vessel; secondly, acupuncture (or laser, or radio frequency) and the like is used to perform membrane rupture and fenestration on the membrane at the opening of the branch vessel; then, dilating the original window by using a balloon; finally, the branch stent 31 is implanted after the dilation of the window is completed, and the window is supported so as to keep the branch blood flow unobstructed.

### Embodiment 1

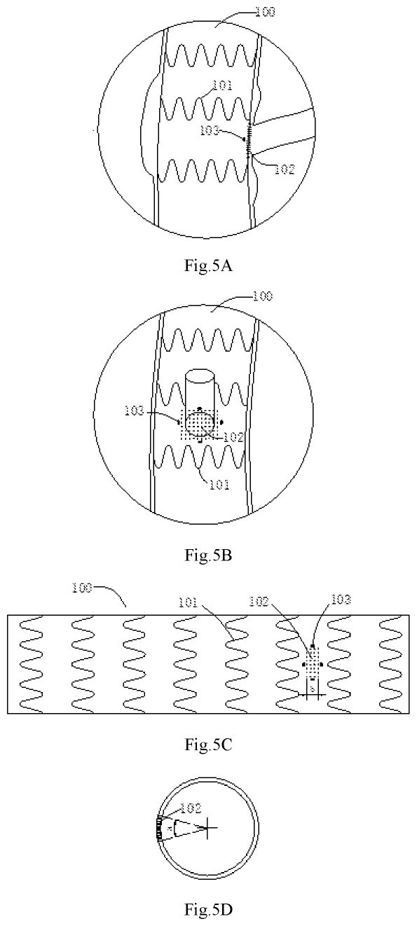

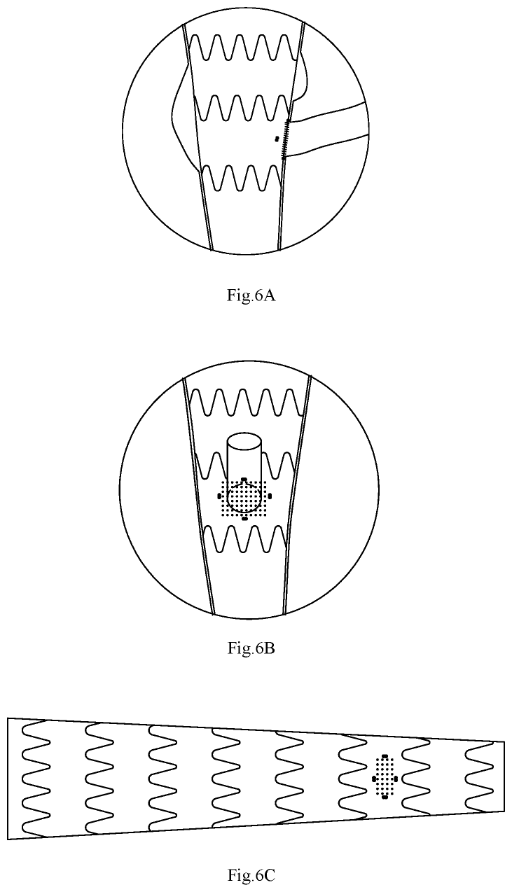

As shown in FIG. 5A-5D, the present application provides a covered stent 100 for implantation at a vascular branch, the covered stent including a stent body 101 and a membrane fixedly connected to the stent body 101, wherein the stent body 101 is a straight cylindrical structure woven from nitinol wires which has sufficient supporting force. A micropore area 102 is provided on the membrane, and the micropore area 102 has a plurality of micropores which are arranged into an array with pore spacing ranging from 0.2 mm to 2.0 mm. The micropores are capable of allowing blood flow to pass through. During implantation of the covered stent 100, the micropore area 102 is used to identify branch region of the blood vessel and provide an access path for implantation of a branch stent.

In one embodiment, the diameters of the plurality of micropores provided in the micropore area 102 are equal, or the diameters of the micropores provided in the middle portion of the micropore areas 102 are larger than those provided in the edge portion of the micropore areas 102. The diameters of the micropores are in the range of 0.1 mm to 0.6 mm. The micropore area 102 is arranged in a range of 15 degrees to 360 degrees along the circumferential direction of the covered stent 100. The length b of the micropore area 102 is in the range of 3 mm to 100 mm.

In one embodiment, the interval between the micropores arranged in the middle portion of the micropore area 102 is smaller than the interval between the micropores arranged in the edge portion of the micropore area 102. The shape of the micropore area 102 include, but is not limited to, a circle, an ellipses, a square, a strip, and the like. The length of the micropore area 102, the diameters of the micropores and the arrangement of the micropores can be adjusted according to the diameter and position of the branch vessel at the lesions. Due to the limitations of the present application on the pore sizes and arrangement of the micropores, as well as the characteristics of high blood flow and high pressure in the aorta, for the micropores that are not located at the opening of the vascular branch attached to the blood vessel wall, after a period of time, blood will accumulate in the micropores causing the micropores to be permanently closed, so there is no need to worry about endoleak.

In one embodiment, a plurality of development marks 103 are provided on the edge of the micropore area 102 to indicate the position of the micropore area 102. The development marks 103 are attached to the edge of the micropore area 102 by precision sewing or riveting. The shapes of the development marks include, but are not limited to, circles, ovals, “8” shapes, and the like. The number of the development marks 103 is 2 to 10. The material of the development marks 103 includes, but is not limited to, platinum, gold, tantalum metal, medical stainless steel, and the like. In the present application, a plurality of development marks 103 are arranged on the edge of the micropore area 102 to indicate the position of the micropore areas 102. The micropore area section of the covered stent can be placed at the opening of the branch vessel through the development marks 103 under the monitoring of Digital Subtraction Angiography (DSA), so that the covered stent will not block the blood flow of the branch vessel while treating aortic lesions. Moreover, the development marks 103 are also beneficial to the positioning of the branch stent, which can be released at the target position after passing through the micropore area 102 to establish blood supply channel of the vascular branch.

In a preferred embodiment, the main stent is partially covered, and the stent at the proximal end portion of the stent body is uncovered. In a preferred embodiment, the main stent is fully covered.

As shown in FIG. 6A-FIG. 6C, in another embodiment, the stent body 101 is a tapered structure formed by laser cutting, and the structure can adapt to the shape characteristics of the human blood vessels (such as carotid arteries) at the implantation site.

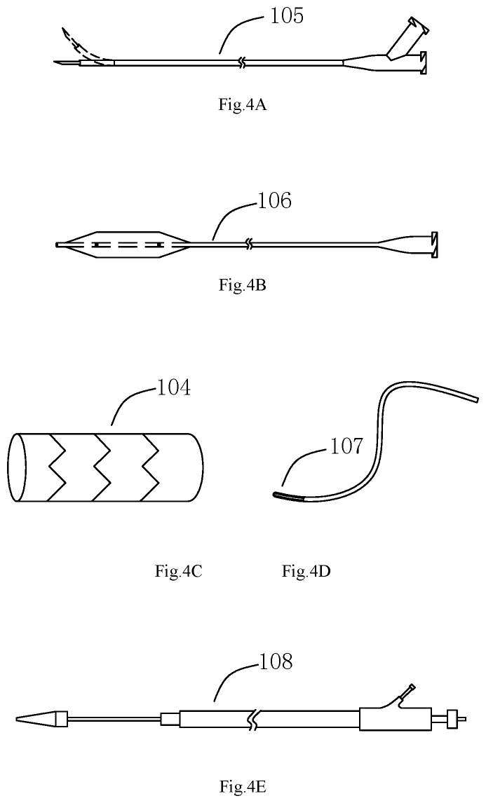

As shown in FIG. 4A-FIG. 4E, the present application also provides a covered stent system for implantation at a vascular branch, the system comprising a delivery system 108, a guide wire 107, and a covered stent provided in the delivery system, a membrane breaker 105, a dilatation balloon 106 and a branch stent 104. As shown in FIGS. 5A-5D, the covered stent 100 includes a stent body 101 and a membrane fixedly connected to the stent body 101. A micropore area 102 is provided on the membrane, and the micropore areas 102 has a plurality of micropores which are arranged into an array with pore spacing in the range of 0.2 mm to 2.0 mm. The micropores are capable of allowing blood flow to pass through. When the covered stent 100 is delivered to the target area by the delivery system and released, the blood flow through the micropores directs the micropore area 102 to be placed at the opening of the vascular branch, so the micropore area 102 can temporarily supply blood to the vascular branch. The membrane breaker 105 is delivered to the micropore area 102 by the delivery system, and the membrane breaker 105 is used to penetrate the micropore area 102 to provide an opening for the dilation balloon 106 to pass through. The dilation balloon 106 is used to further increase the size of the opening to facilitate the support of the branch stent 104 through the micropore area 102 at the vascular branch to establish the blood supply channel of the vascular branch.

In this embodiment, the delivery system 108, the membrane breaker 105, the dilation balloon 106 and the branch stent 104 may all use the structures commonly used in the prior art, and the detailed structures thereof will not be repeated herein again.

In this embodiment, the material of the stent body 101 includes, but is not limited to, nickel-titanium alloy, cobalt-chromium alloy, magnesium alloy, 316L stainless steel, metal tantalum, and polymer materials. The membrane is biocompatible, and is fixed to the stent body 101 by precision sewing or thermoforming. The material of the membrane includes but is not limited to polytetrafluoroethylene, polyester, polyethylene terephthalate, polyurethane esters, etc.

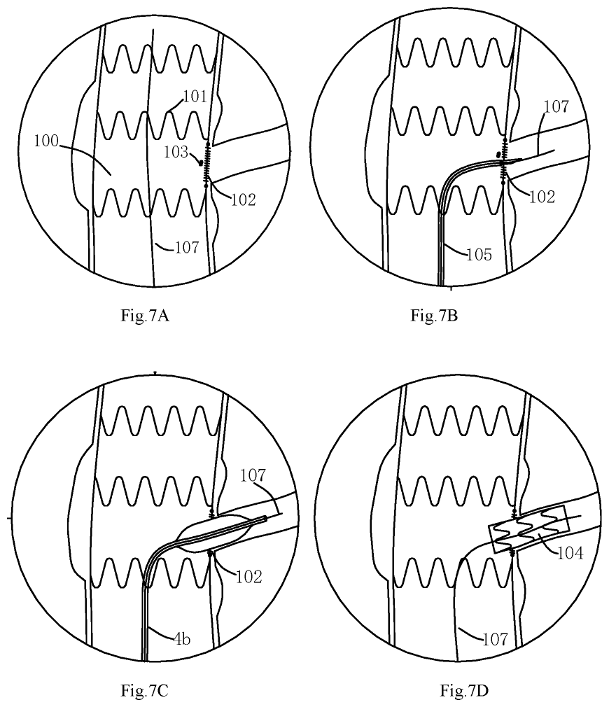

As shown in FIGS. 7A-7D, the operation steps of the covered stent system of the present application are as follows:

Firstly, as shown in 7a, under the monitoring of digital subtraction angiography (DSA), the delivery system loaded with the covered stent 100 is delivered to the diseased blood vessel along the guide wire 107. Under the condition that the blood flow in the visible micropore area 102 passes through and/or under the indication of the development marks 103, the micropore area 102 are placed at the opening of the branch vessel, and the covered stent is released after accurately aligning. At this time, the covered stent not only covers the location of the arterial lesions, but also ensures the blood supply of the branch vessel due to the permeability of the micropore area 102 to blood flow.

Secondly, as shown in FIG. 7B, the membrane breaker 105 is transported along the guide wire 107 to the opening of the branch vessel where the micropore area 102 is located, and the membrane perforation is performed to form an opening, and then the membrane breaker 105 is withdrawn after completion. Since the presence of the micropore area 102 reduces the transmembrane puncture force, the transmembrane puncture also becomes easier.

Thirdly, as shown in FIG. 7C, the dilation balloon is transported along the guide wire 107 to the opening to further increase the size of the opening. Since the window dilation is performed in the micropore area 102, the risk of membrane tear at the window caused by balloon dilation is reduced to a certain extent.

Fourthly, as shown in FIG. 7D, the branch stent 104 is implanted along the guide wire 107 at the opening of the branch vessel to support and stabilize the window, so as to keep the blood flow of the branch unobstructed.

It can be seen from the above description that the present application does not adopt the “chimney” technique that is prone to endoleak. The covered stent and covered stent system provided by the present application are provided with a micropore area on the membrane, and the micropore area has a plurality of micropores, which are capable of allowing blood flow to pass through. During the implantation of the covered stent, the micropore area can identify the vascular branch area and temporarily supply blood to the vascular branch, so the operation is simple and does not require establishing extracorporeal circulation, the operation time is short and the difficulty is low. Moreover, a plurality of micropores arranged in the micropore area are arranged into an array with pore spacing in the range of 0.2 mm to 2.0 mm, and the diameters of the micropores in the range of 0.1 mm to 0.6 mm. The setting of the micropore diameters and pore spacing can not only ensure the blood supply of the branch vessel, but also prevent the membrane from tearing during the puncture of the membrane breaker. Even if the dilation balloon is further dilated, it only changes the size of the adjacent micropores in the micropore area, but will not tear the membrane. Therefore, the micropore area can provide a good access indication and access window for the implantation of the branch stent, which not only solves the problems of difficult positioning and operation of the existing “branch stent” technique, but also solves the problems of serious surgical complications and complex surgery of the “fenestration” technique.

### Embodiment 2

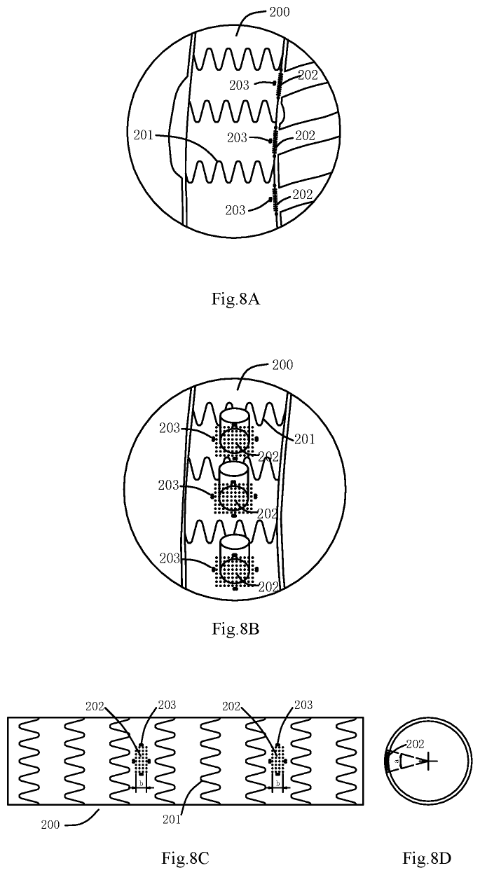

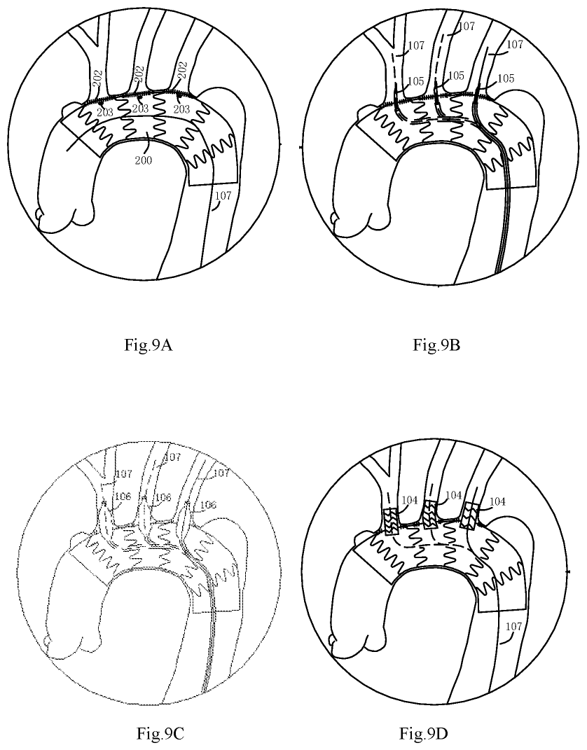

As shown in FIGS. 8A to 8D, a covered stent 200 for implantation at vascular branches, includes a stent body 201 and a membrane fixedly connected to the stent body 201. The stent body 201 is a straight cylindrical structure woven from nitinol wires and has sufficient supporting force. The difference between this Embodiment and Embodiment 1 is that a plurality of micropore areas 202 are provided on the membrane, which can be respectively placed at different branch vessel openings. This structure is suitable for the situation where there are multiple branch vessels at the lesion location. The setting of the pore diameters and the pore spacing of the plurality of micropores in the micropore areas 202 can be adjusted correspondingly according to the diameters and position of the branch vessels at the lesion. In one embodiment, a plurality of development marks 203 are provided on the edges of the plurality of micropore areas 202 to indicate the positions of the micropore areas 102.

FIGS. 9A-9D show the operation steps of the covered stent system of this embodiment.

The difference between this Embodiment and Embodiment 1 is that multiple micropore areas 202 need to be placed at different branch vessel openings, respectively to ensure the accuracy of the alignment and smooth blood flow of each branch vessel, and then repeat steps 2 to 4 several times respectively.

### Embodiment 3

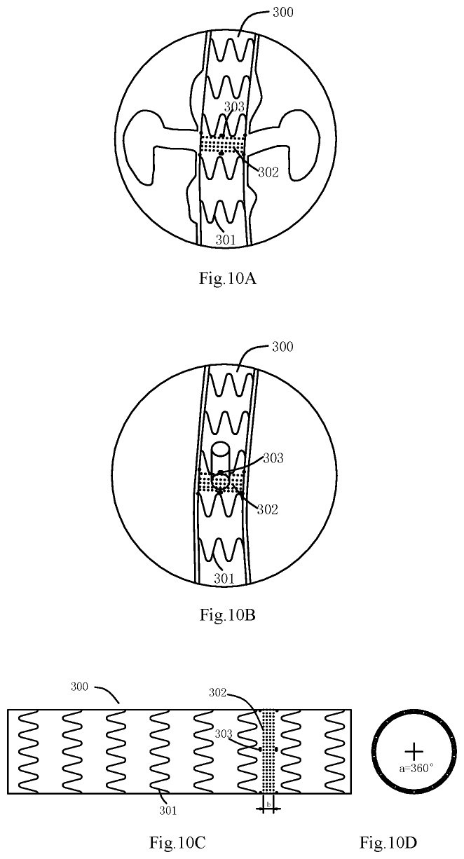

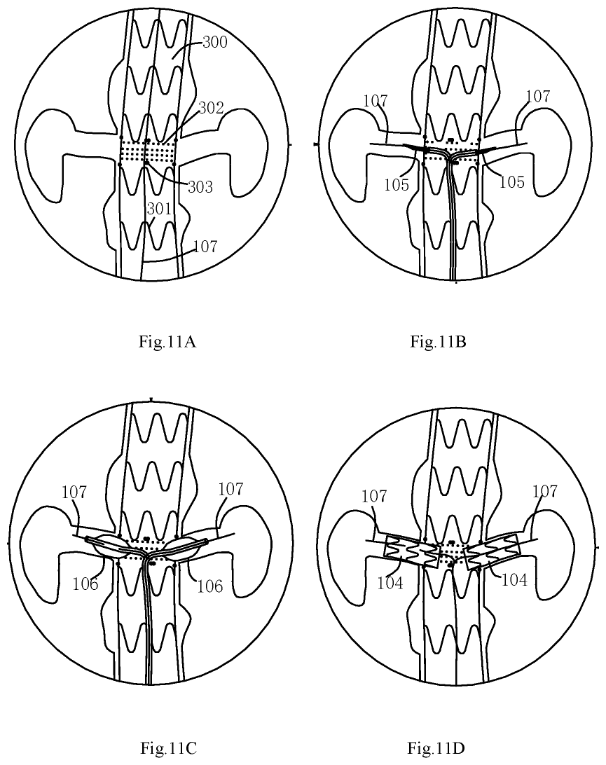

As shown in FIGS. 10A-10D, a covered 300 for implantation at vascular branches, includes a stent body 301 and a membrane fixedly connected to the stent body 301. The stent body 301 is a straight cylindrical structure woven from nitinol wires, which has sufficient supporting force. The difference between this embodiment and Embodiment 1 is that a certain area of the membrane is provided with micropores 360 degree along the circumferential direction of the stent body 301, that is, a micropore area 302 is provided 360 degree along the circumferential direction of the covered stent. The structure of the covered stent 300 is suitable for the case where there are multiple branch vessels in the same radial direction at the lesion location, such as renal artery vascular lesions.

FIGS. 11A-11D show the operation steps of the covered stent system of this embodiment. The difference between this Embodiment and Embodiment 1 is that development marks 303 are provided on the edge of the micropore area 302. Under the indication of the development marks 303, the micropore area 302 is placed at the opening of the branch vessels. At this time, it is not necessary to consider the radial position of the micropore area 302. The membrane is provided with micropores at 360 degrees in a certain region, which can not only adapt to the situation that there are multiple branch vessels in the same radial direction at the lesion location, but also reduce the difficulty for the operator to align the micropore area 302 with the openings of the branch vessels. In addition, due to the limitation of the micropore diameters and the characteristics of fast blood flow and high pressure in the arteries, after a period of time, blood accumulation will occur in the micropores that are not located at the openings of the vascular branches in the same radial direction, resulting in permanent closure of the micropores, so there is no need to worry about endoleak.

### Embodiment 4

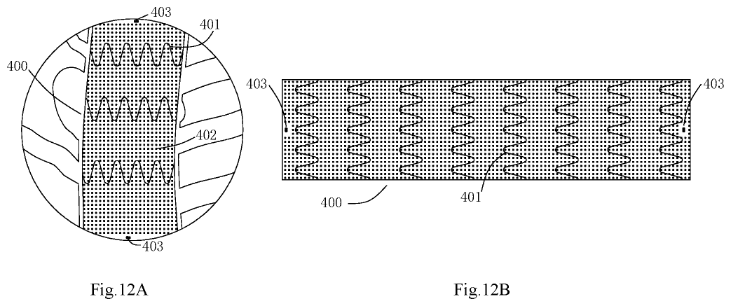

As shown in FIG. 12A and FIG. 12B, a covered stent 400 for implantation at vascular branches, includes a stent body 401 and a membrane fixedly connected to the stent body 401. The stent body 401 is a straight cylindrical structure woven from nitinol wires and has sufficient supporting force. Different from Embodiment 1, the covered stent in this embodiment is provided with a micropore area 402 on the entire membrane. The covered stent involved in this embodiment is suitable for cases with complex branches and numerous branches in aortic lesions, and the structure will not block the blood supply of the branch vessels.

In this embodiment, the difference of the operation steps between this embodiment and Embodiment 1 is that the two ends of the covered stent are provided with development marks 403. Under the indication of the development marks 403, the covered stent 400 is placed at the aortic lesions through a delivery system, subsequent operations such as hole breaking, dilating, and implantation of branch stents can be performed. Since there are micropores on the entire membrane, there is no problem of aligning the micropore area 403 with the openings of the branch vessels, so there is no need to worry about the membrane blocking the branch vessels. In addition, due to the limitation of the micropore diameters and the characteristics of high blood flow rate and high pressure in the aorta, for the micropores that are not located at the openings of the vascular branches, after a period of time, blood accumulation will appear in the micropores, causing the micropores to be permanently closed, so there is no need to worry about endoleak.

In the specific application process, the appropriate number and range of micropore areas can be set according to the anatomical structure of the lesion area, and the micropore areas can be set within the range of 15 degrees to 360 degrees along the circumferential direction of the covered stent.

The above detailed description of the application aims to enable those skilled in the art to understand the content of the application and implement it. It does not limit the scope of protection of the application. Any equivalent changes or modifications made according to the spirit of the application should be covered in the scope of protection of the application.

1. A covered stent for implantation at vascular branches, comprising a stent body and a membrane fixedly connected to the stent body, wherein micropore areas are provided on the membrane, and the micropore areas have a plurality of micropores which are arranged into an array with pore spacing ranging from 0.2 mm to 2.0 mm, and the micropores are capable of allowing blood flow to pass through, and during implantation of the covered stent, the micropore areas are used to identify branch regions of blood vessels and provide an access path for implantation of branch stents.

2. The covered stent for implantation at vascular branches according to claim 1, wherein diameters of the micropores are in the range of 0.1 mm to 0.6 mm, and the lengths of the micropore areas are in the range of 3 mm to 100 mm.

3. The covered stent for implantation at vascular branches according to claim 2, wherein the membrane is provided with one or more micropore areas, and the micropore areas are arranged in a range of 15 degrees to 360 degrees along the circumferential direction of the covered stent.

4. The covered stent for implantation at vascular branches according to claim 1, wherein a plurality of development marks are provided on the edge of the micropore areas to indicate the position of the micropore areas.

5. The covered stent for implantation at vascular branches according to claim 4, wherein the development marks are attached to the edge of the micropore areas by one of precision sewing and riveting.

6. A covered stent system for implantation at vascular branches, comprising a delivery system and a covered stent provided in the delivery system, a membrane breaker, a dilatation balloon and branch stents, and the covered stent comprises a stent body and a membrane fixedly connected to the stent body, micropore areas are provided on the membrane, and the micropore areas have a plurality of micropores which are arranged into an array with pore spacing ranging from 0.2 mm to 2.0 mm, and the micropores are capable of allowing blood flow to pass through, and when the covered stent is delivered to the target area by the delivery system and released, the blood flow through the micropores directs the micropore areas to be placed at the opening of the vascular branch, so the micropore areas can temporarily supply blood to the vascular branch, and the membrane breaker is used to penetrate the micropore areas to provide an opening for the dilation balloon to pass through, and the dilation balloon is used to further increase the size of the opening to facilitate the support of the branch stents through the micropore areas at the vascular branches to establish the blood supply channels of the vascular branches.

7. The covered stent system for implantation at vascular branches according to claim 6, wherein the diameters of the micropores are in the range of 0.1 mm to 0.6 mm, and the lengths of the micropore areas are in the range of 3 mm to 100 mm.

8. The covered stent system for implantation at vascular branches according to claim 7, wherein the membrane is provided with one or more micropore areas, and the micropores areas are arranged in a range of 15 degrees to 360 degrees along the circumferential direction of the covered stent.

9. The covered stent system for implantation at vascular branches according to claim 7, wherein a plurality of development marks are provided on the edge of the micropore areas to indicate the position of the micropore areas.

10. The covered stent system for implantation at vascular branches according to claim 9, wherein the development marks are attached to the edge of the micropore areas by one of precision sewing and riveting.
